(19) United States
(12) Patent Application Publication
(10) Pub. No.: US 2011/0223166 A1
(43) Pub. Date: Sep. 15, 2011

(54) Methods for Preventing Pressure-Induced Apoptotic Neural-Cell Death

(75) Inventor: Minas Theodore Coroneo, Randwick (AU)

(73) Assignee: MINAS THEODORE CORONEO, RANDWICK (AU)

(21) Appl. No.: 13/088,550

(22) Filed: Apr. 18, 2011

Related U.S. Application Data
(63) Continuation of application No. 11/171,531, filed on Jun. 30, 2005, now abandoned.
Application No. 11/171,531 is a continuation-in-part of application No. 10/084,604, filed on Feb. 27, 2002, now abandoned.
Application No. 10/084,604 is a continuation of application No. 09/649,643, filed on Aug. 29, 2000, now abandoned.

(51) Int. Cl. A61K 39/395; A61P 27/06

(52) U.S. Cl. 424/135.1

(57) ABSTRACT

Compositions and methods for protecting neuronal cells from pressure-induced apoptotic cell death which comprises administering to a subject in need of such treatment at least one compound which directly or indirectly inhibits the activity of an ion channel on neuronal cells and thereby inhibits the effect of pressure on the cells.

The present application is a continuation-in-part application of U.S. patent application Ser. No. 10/084,604 filed Feb. 27, 2002, which is a continuation of 09/649,643 filed Aug. 29, 2000. The entire text of each of the aforementioned applications is incorporated herein by reference.

### FIELD OF THE INVENTION

This invention is concerned with methods and compositions for protecting neural tissue from cell death, more particularly, apoptotic cell death associated with elevated pressure. In a further aspect the invention is concerned with methods and compositions for the treatment or prevention of pressure-induced damage to neuronal cells such as occurs in glaucoma, or damage to neuronal cells of the central nervous system resulting from elevated pressure in the CNS, and peripheral nerve damage associated with elevated pressure.

### BACKGROUND OF THE INVENTION

Neuronal tissue or nerve cell death is a major medical problem in human society. Neuronal cell death in the eye may lead to blindness. Glaucoma is a principal cause of neural cell apoptotic death in the eye and a principal cause of adult blindness. It is the third major cause of visual loss in the elderly, affecting approximately 3% of the population over 50.

Neuronal cell death is associated with a range of other medical conditions. These include hydrocephalus, and other brain/skull diseases or injuries. Brain neuron cell death may result in mental impairment, loss of motor functions and the like.

Peripheral nerve damage from traumatic injury or surgical complications, for example, in the spine, feet and hands may cause apoptotic cell death. In the spinal column spinal bones may press upon a nerve trunk causing nerve cell death (eg in spinal stenosis). Bone and connective tissue pressure on nerves in peripheral tissue such as the wrists may cause apoptotic neural cell death in the median nerve and consequent lack of feeling and/or motor movement (eg in carpel tunnel syndrome).

Morphologically apoptosis is characterised by progressive condensation of the cytoplasm and nucleus, followed by fragmentation and phagocytosis by other cells (Majino and Joris (1995) Am Pathol 146: 3-15).

Although there are some known inhibitors of apoptosis, there are no effective therapeutic agents for the treatment of pressure-induced apoptotic neuronal cell death.

In relation to glaucoma, there are now a number of agents which reduce intraocular pressure, with mixed success. The mechanism of action of such agents is controversial and unclear.

Apoptotic neural cell death in the central nervous system (CNS) is associated with wide range of further conditions. For example, elevated pressure in the central nervous system may result from conditions such as space-occupying lesions (eg tumors), which cause compression of venous sinuses and therefore prevention of cerebrospinal fluid (CSF) absorption in the arachnoid villi. Elevated CSF pressure also occurs in cerebral edema, usually associated with brain injury, hydrocephalus and inflammatory lesions and, spinal compression.

Conditions associated with elevated neuronal cell pressure remain significant problems, with no effective therapeutic agents being available.

### SUMMARY OF THE INVENTION

In a first aspect of this invention, there is provided a method for the treatment or prevention of a condition associated with pressure-induced apoptotic neuronal cell death which comprises administering to a subject in need of such treatment at least one compound which directly or indirectly inhibits activity of a stretch-activated ion channel on neuronal cells.

In a further form, the present invention relates to a method for the treatment or prevention of apoptotic ocular nerve cell damage in glaucoma resulting from elevated intraocular pressure which comprises administering to a subject in need of such treatment at least one compound which directly or indirectly inhibits activity of a stretch-activated ion channel on ocular neuronal cells.

In yet another form, the present invention relates to a method for the treatment or prevention of apoptotic damage to neuronal cells of the central nervous system resulting from elevated pressure in the central nervous system which comprises administering to a subject in need of such treatment at least one compound which directly or indirectly inhibits activity of a stretch-activated ion channel on CNS neuronal cells.

In still yet another form, the present invention relates to a method for the treatment or prevention of apoptotic peripheral nerve damage resulting from elevated pressure in peripheral nervous system which comprises administering to a subject in need of such treatment at least one compound which directly or indirectly inhibits activity of a stretch-activated ion channel on peripheral neuronal cells.

In another aspect, the present invention provides a composition for the treatment or prevention of a condition associated with pressure-induced apoptotic neuronal cell death which comprises at least one compound which inhibits activity of the effects of pressure on neuronal cells by directly or indirectly inhibiting activity of a stretch-activated ion channel, optionally in association with one or more pharmaceutically acceptable carriers or excipients.

In a further form, the present invention relates to a composition for the treatment or prevention of apoptotic ocular nerve cell damage in glaucoma resulting from elevated intraocular pressure which comprises at least one compound which directly or indirectly inhibits activity of a stretch-activated ion channel on ocular neuronal cells, optionally in association with one or more pharmaceutically acceptable carriers or excipients.

In still yet another form, the present invention relates to a composition for the treatment or prevention of apoptotic damage to neuronal cells of the central nervous system resulting from elevated pressure in the CNS which comprises at least one compound which directly or indirectly inhibits activity of a stretch-activated ion channel on CNS neuronal cells, optionally in association with one or more pharmaceutically acceptable carriers or excipients.

Another aspect of the invention relates to a composition for the treatment of apoptotic peripheral nerve damage resulting from elevated pressure in the peripheral nervous system which comprises at least one compound which directly or indirectly inhibits activity of a stretch-activated ion channel on peripheral neuronal cells, optionally in association with one or more pharmaceutically acceptable carriers or excipients.

Preferably, the at least one compound which directly or indirectly inhibits activity of a stretch-activated ion channel on neuronal cells is identified by patch clamping.

Even more preferably, the stretch-activated ion channel inhibited on neuronal cells in the methods and compositions of the invention is a potassium channel.

In a particularly preferred form, the stretch-activated ion channel is TREK-1 or TRAAK.

In a preferred form, the stretch-activated ion channel is TREK-1 and the at least one compound which inhibits activity of TREK-1 is a cationic amphipathic compound.

Preferably, the at least one compound includes a six-membered ring structure having at least two heteroatoms, wherein at least one of the heteroatoms is a nitrogen atom, such compounds including for example as sipatrigine, amiloride, chlopromazine or methochlorpromazine, or analogues thereof.

In another form, the at least one compound is serotonin, gentamicin, mibefradil, tetracaine, GsTMx-4, quinine, quinidine, imipramine, caffeine, theophylline, a PDE-IV inhibitor, an antisense TREK-1 polynucleotide, an antisense TRAAK polynucleotide, an anti-TREK-1 antibody, an antibody against a TREK-1 or TRAAK effector molecule, an anti-PIP₂ antibody or propranol, or analogues thereof.

### DETAILED DESCRIPTION OF THE INVENTION

This invention provides methods and compositions for the treatment or prevention of conditions associated with pressure-induced apoptotic neuronal cell death. The invention is based on the surprising finding that elevated pressure on neuronal cells induces apoptotic cell death. The invention is also based on the unexpected finding that compounds which inhibit the activity of stretch-activated channels in neuronal cells protect the neuronal cells against pressure induced apoptotic cell death.

The effects of pressure on neuronal cells may be blocked though the use of compounds which inhibit the activity of a stretch-activated ion channel on neuronal cells.

Stretch-activated channels have been described by various authors, and may be regarded as being associated with mechanoelectric transduction (see Zeng et al (2000) Heart and Circulatory Physiology 278 (2): H548). Stretch-activated channels (SACs) are found in a variety of cells including cardiomyocytes (see Hu and Sachs (1996) J Membr Biol 154: 205-216). Examples of potassium ion channels include stretch activated channels from of a family of two-pore domain K⁺ channels (K_{2P}). Eight two-pore domain potassium channels have been cloned in rodents and humans. There are 4 classes:
TWIK-1 & TWIK-2 (Tandem of P domains in Weak Inward rectifier K⁺ channels) are weak inward rectifiers;
TREK-1 (TWIK-Related K⁺ channel) & TRAAK (TWIK-related Arachidonic Acid (AA)-stimulated K⁺ channel) are polyunsaturated fatty acids (FA) and are also stretch-activated K⁺ channels (see Meadows et al, Brain Research 2001; 892, 94-101);
TASK-1 and TASK-2 (TWIK-related Acid-Sensitive K⁺ channels) are acid-sensitive K⁺ channels;
KCNK6 and KCNK7 are silent subunits that probably need a partner to become active.

(see Maingret et al, J Biol Chem. 2000;275:10128-33).

Of the stretch-activated potassium channels, TRAAK (SEQ ID NO:2) appears to be restricted to the central nervous system, spinal cord and retina. TREK-1 (SEQ ID NO:1) is ubiquitous with strong expression in the central nervous system. Both TREK-1 and TRAAK are outward rectifier K⁺ channels opened by membrane stretch, cell swelling, and/or shear stress (all pressure effects). At atmospheric pressure, basal activity is negligible and channels are opened by convex curvature of the plasma membrane. Mechano-gating does not require the integrity of the cytoskeleton and the activating force is apparently directly coming from the cell membrane bilayer. Cytoskeleton disruption potentiates the opening by membrane stretch, suggesting that these channels are tonically repressed by the cytoskeleton.

Inhibiting the activity of stretch-activated ion channels may be measured according to conventional physiological techniques, such as by voltage clamp recordings (patch clamping) from isolated cells subject to membrane stretching, for example resulting from increased pressure or induced physical stretching, such as subjecting isolated cells to controlled strain such as longitudinal stretch. Under these conditions, stretch-activated channels may be measured by elicited electrical current. The elicited current may represent inward cationic currents such as described by Zeng et al (2000) Heart and Circulatory Physiology 278 (2): H548, or outward cationic currents.

Suitable patch-clamping methods are well known in the art, and include, for example, for a single-channel patch clamp, patch voltage may be controlled by an Axopatch 200B (Axon Instruments) and stored directly on computer disk using, for example, a Labmaster DMA version B (Scientific Instruments) board controlled by pClamp6-Clampex acquisition software (Axon Instruments). Currents may be sampled at 10 kHz and low-pass filtered at 2 kHz through a four-pole Bessel filter on the Axopatch 200B. Experimental voltage protocols may be controlled by pClamp6-Clampex. Potentials are routinely defined with respect to the extracellular surface. Electrodes can be pulled on a pipette puller (eg PC-84; Brown-Flaming Instruments), painted with Sylgard 184 (Dow Corning Corp.) and typically fire polished.

For potassium stretch-activated channels, electrodes are typically filled with KCl saline containing (mM): 140 KCl, 5 EGTA, 2 MgSO4, 10 HEPES, pH 7.3). Bath saline typically consists of (mM): 140 NaCl, 5 KCl, 1 MgSO4, 1 CaCl2, 6 glucose, and 10 HEPES, pH 7.3.

Pressure and suction can be applied to the pipette by a pressure clamp. The rise time of pressure changes at the tip can be determined by monitoring the rate of current change when pressure steps are applied to an electrode containing 150 mM KCl solution and placed in a water bath. Perfusion of a patch may be handled by a pressurized bath perfusion system with eight separate channels (BPS-8; ALA Scientific). Offline data analysis can be performed with pClamp6 analysis software and Origin 5.0. Maximal unitary channel currents can be determined via Gaussian fits to the peaks of the all-points amplitude histograms from records containing one to three channels.

Whole-cell currents can be measured by the Nystatin-perforated patch technique (Horn & Marty, J Gen Physiol, 1988, 92:145-59). Bath saline is typically the same as that for single patch clamping, above. Pipette saline is typically 80 mM KCl, 30 mM K₂SO₄, 10 mM NaCl, 3 mM MgSO₄, 0.13 mM CaCl₂, 0.23 mM EGTA, and 10 mM HEPES at pH 7.3. Nystatin is typically dissolved in pipette saline to a final concentration of 200 μg/ml. Access resistance is allowed to drop after patch formation, then series resistance compensation is set. Whole-cell currents may be measured by a voltage-step protocol or a voltage-ramp protocol.

The compound of interest (ie the potential inhibitor of the stretch-activated ion channel) is then typically applied rapidly to a cell or patch of interest by gravity flow through a local perfusion device using two-barrel theta tubing, or by pressurized bath perfusion systems for a single patch (eg BPS-8; ALA Scientific), and any changes in current are subsequently recorded.

Agents which inhibit stretch-activated channels reduce or abolish the elicited currents. Preferably stretch induced currents are reduced by the compounds, for example, by between 10% and 100%, such as 10% and 90%, 10% and 80%, 10% and 70%, 10% and 60%, 10% and 50%, 10% and 40%, 10% and 30%, and 10% and 20%.

In another aspect this invention is concerned with the method for the treatment or prevention of a condition associated with pressure-induced apoptotic neuronal cell death which comprises administering to a subject in need of such treatment at least one compound which directly or indirectly inhibits activity of a stretch-activated ion channel on neuronal cells.

The term “a condition associated with pressure-induced apoptotic neuronal cell death” as used herein refers to a pathological state characterized by, or otherwise associated with, elevated pressure surrounding neuronal cells in a particular area of the nervous system. The pressure-induced apoptotic neuronal cell death may be primary (ie directly involved in the etiology of the condition, such as occurs in glaucoma) or secondary to the condition (ie indirectly involved or consequential to another condition, such as occurs in compression of a spinal nerve due to a collapsed intervertebral disc).

In another aspect of the invention there is provided a method for the treatment of apoptotic ocular nerve cell damage in glaucoma resulting from elevated intraocular pressure which comprises administering to a subject in need of such treatment at least one compound which directly or indirectly inhibits activity of a stretch-activated ion channel on ocular neuronal cells.

In another aspect of the invention there is provided a method for the treatment or prevention of apoptotic damage to neuronal cells of the CNS resulting from elevated pressure in the CNS which comprises administering to a subject in need of such treatment at least one compound which directly or indirectly inhibits activity of a stretch-activated ion channel.

In a another aspect of the invention there is provided a method for the treatment or prevention of apoptotic peripheral nerve damage resulting from elevated pressure in the peripheral nervous system which comprises administering to a subject in need of such treatment at least one compound which directly or indirectly inhibits activity of a stretch-activated ion channel on peripheral nerve cells.

In a further aspect of the invention there is provided a composition for the treatment or prevention of a condition associated with pressure-induced apoptotic cell death which comprises at least one compound which directly or indirectly inhibits activity of a stretch-activated ion channel on neuronal cells, optionally in association with one or more pharmaceutically acceptable carriers or excipients.

In a further aspect of the invention there is provided a composition for the treatment of apoptotic ocular nerve cell damage in glaucoma resulting from elevated intraocular pressure which comprises at least one compound which directly or indirectly inhibits activity of a stretch-activated ion channel, optionally in association with one or more pharmaceutically acceptable carriers or excipients.

In a further aspect of the invention there is provided a composition for the treatment or prevention of apoptotic damage to neuronal cells of the CNS resulting from elevated pressure in the CNS which comprises at least one compound which directly or indirectly inhibits activity of a stretch-activated ion channel, optionally in association with one or more pharmaceutically acceptable carriers or excipients.

In a further aspect of the invention there is provided a composition for the treatment or prevention of apoptotic peripheral nerve damage resulting from elevated pressure in the peripheral nervous system which comprises at least one compound which directly or indirectly inhibits activity of a stretch-activated ion channel, optionally in association with one or more pharmaceutically acceptable carriers or excipients.

Compounds which block the apoptotic effect of pressure on neuronal cells, by directly or indirectly inhibiting activity of stretch-activated ion channels include: sodium ion channel blockers, calcium ion channel blockers, potassium channel blockers.

The compounds suitable for use in the methods and compositions of the invention may: act directly on a stretch-activated ion channel on neuronal cells to inactivate the channel, or may act indirectly on the stretch-activated ion channel, such as by activating or inhibiting, respectively, one or more other physiological molecules that inhibit or activate, respectively, the activity of the stretch-activated ion channel.

The term “effector molecules” as used herein refers to physiological molecules that activate or inhibit the activity of stretch-activated potassium channels.

The compounds may include antibodies to the stretch-activated ion channels. It has been found that the mechanism of action of the stretch-activated potassium channels, TREK-1, TREK-2 and TRAAK is mediated in part by 4,5-bisphosphate (PIP₂), and that an antibody directed against PIP₂ reduces current levels by competing with the channels for PIP₂ (Lopes et al., J Physiol, 2005, 564(1): 117-129). Accordingly, PIP₂ is an example of an effector molecule.

The term “antibodies” as used herein encompasses polyclonal and monoclonal antibodies, chimeric, single-chain and humanized antibodies, as well as Fab fragments, including the products of a Fab or other immunoglobulin expression library.

The stretch-activated ion channel polypeptides that are inhibited or blocked by the compositions and methods of the invention, or their fragments or analogs thereof, or cells expressing them, can also be used as immunogens to produce antibodies immunospecific for stretch-activated ion channels.

The term “immunospecific” as used herein means that the antibodies have substantially greater affinity for stretch-activated ion channels than their affinity for other related polypeptides.

Antibodies generated against the stretch-activated ion channels may be obtained by administering the polypeptides or epitope-bearing fragments, analogs or cells to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., Monoclonal Antibodies and Cancer Therapy, 77-96, Alan R. Liss, Inc., 1985).

Techniques for the production of single chain antibodies, such as those described in U.S. Pat. No. 4,946,778, can also be adapted to produce single chain antibodies to the stretch-activated ion channels. Also, transgenic mice, or other organisms, including other mammals, may be used to express humanized antibodies.

Particularly preferred for the methods and compositions of the present invention are antibodies raised against stretch-activated potassium channels, and in particular, anti-TREK-1 or anti-TRAAK antibodies.

Alternative therapeutic compounds for the methods and compositions of the invention are isolated nucleic acid molecules which are antisense to polynucleotides encoding stretch-activated ion channels. An “antisense” nucleic acid comprises a nucleotide sequence which is complementary to a “sense” nucleic acid encoding a protein, eg complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire neuronal stretch-activated ion channel coding strand, or to only a portion thereof. In one form, an antisense nucleic acid molecule may be antisense to a “coding region” of the coding strand of a nucleotide sequence encoding a neuronal stretch-activated ion channel.

The term “coding region” refers to the region of the nucleotide sequence comprising codons which are translated into amino acid residues.

Alternatively, the antisense nucleic acid molecule may be antisense to a “noncoding region” of the coding strand of a nucleotide sequence encoding the neuronal stretch-activated ion channel. The term “noncoding region” refers to 5′ and 3′ sequences which flank the coding region that are not translated into amino acids (ie also referred to as 5′ and 3′ untranslated regions).

Given the coding strand sequences encoding the stretch-activated ion channels inhibited by the methods and compositions of the invention, antisense nucleic acids can be designed according to the rules of Watson and Crick base pairing. The antisense nucleic acid molecule can be complementary to the entire coding region of neuronal stretch-activated ion channel mRNA, but more preferably is an oligonucleotide which is antisense to only a portion of the coding or noncoding region of neuronal stretch-activated ion channel mRNA. For example, the antisense oligonucleotide can be complementary to the region surrounding the translation start site of neuronal stretch-activated ion channel mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (eg an antisense oligonucleotide) can be chemically synthesized using naturally occurring nucleotides or variously modified nucleotides designed to increase the biological stability of the molecules or to increase the physical stability of the duplex formed between the antisense and sense nucleic acids, eg phosphorothioate derivatives and acridine substituted nucleotides can be used. Such polynucleotides are referred to as polynucleotide analogues.

In terms of polynucleotides, the term “analogue” as used herein refers to a polynucleotide which does not have exactly the nucleotide sequence as a given antisense polynucleotide, but which still is capable of mediating contact with an mRNA polynucleotide encoding a stretch-activated ion channel. Generally, such polynucleotides will be polynucleotides which vary eg to a certain extent in the polynucleotide sequence by way of conservative polynucleotide substitution, and/or incorporation of chemically modified polynucleotides.

Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (ie RNA transcribed from the inserted nucleic acid will be of an antisense orientation to a target nucleic acid of interest.

The antisense nucleic acid molecules suitable for use in the methods and compositions of the invention are typically administered to a subject or generated in situ such that they hybridize with or bind to cellular mRNA and/or genomic DNA encoding a neuronal stretch-activated ion channel protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, eg by linking the antisense nucleic acid molecules to peptides or antibodies which bind to cell surface receptors or antigens.

Particularly preferred antisense nucleotides for the methods and compositions of the present invention are stretch-activated antisense polynucleotides, and in particular, TREK-1 or TRAAK antisense polynucleotides.

At least one active compound is used in the compositions and methods of the invention. For example, two or more compounds may be used in combination. Such combinations may involve synergistic interactions. The effects may occur directly and/or indirectly on the stretch-activated ion channels.

Suitable compounds can be readily identified by testing apoptotic protecting activity under pressure, whether, for example, under atmospheric or hydrostatic pressure or such as by physical stretching of cells. Where neuronal cells are subjected to elevated pressure, such as 100 mm Hg for two hours or more, pressure induced apoptotic cell death occurs, as can be determined by apoptosis assays (see Agar A et al J Neurosci Res 2000; 60: 495-503).

As discussed, compounds which inhibit the activity of a stretch-activated ion channel on neuronal cells can be readily-identified by conventional physiological techniques, such as patch/voltage clamp recordings from isolated neuronal cells subject to elevated pressure as described above (Zeng et al (2000) Heat and Circulatory Physiology 278(2):H548). Compounds which inhibit elicited currents may be used in this invention.

Particularly preferred compounds for the compositions and methods of the invention are those that inhibit activity of neuronal stretch-activated potassium channels. Such compounds include, but are not limited to; those including a six-membered ring:

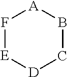

wherein the ring has at least two heteroatoms located at any two positions of A, B, C, D, E, or F, wherein at least one of the two heteroatoms is nitrogen. Exemplary compounds include, but are not limited to:

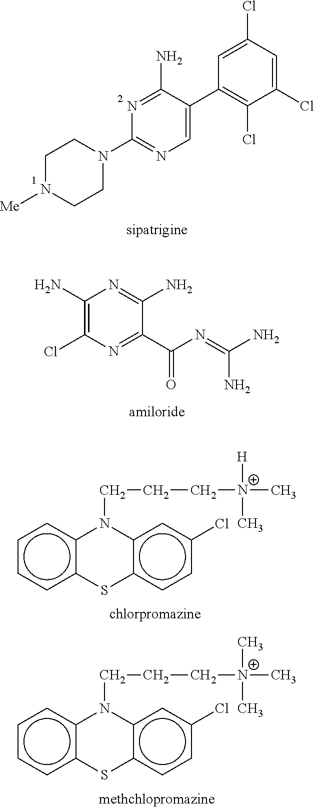

Additionally, compounds that block neuronal stretch-activated potassium channels include:

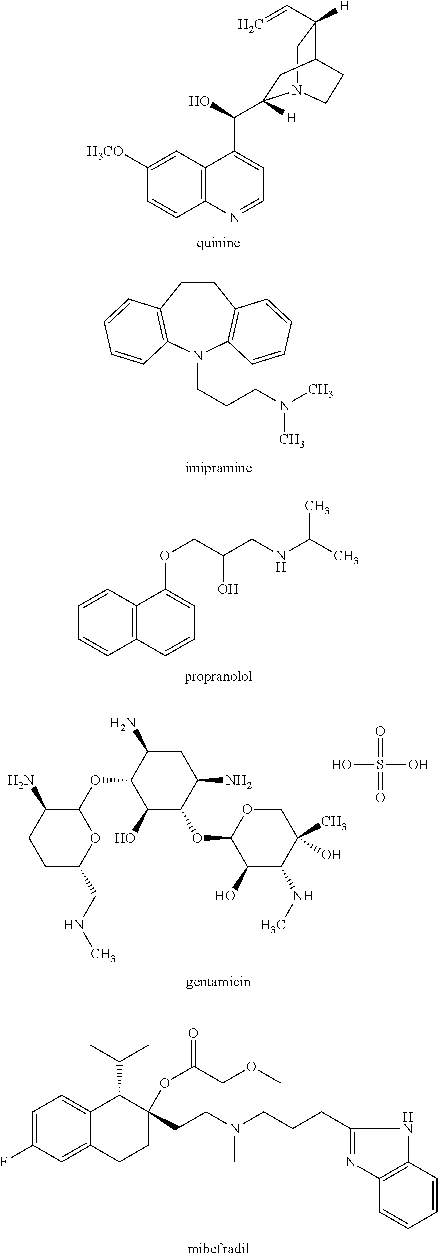

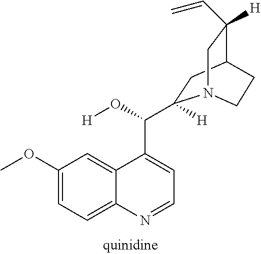

TREK-1 activity is inhibited by serotonin by cAMP-induced phosphorylation (Patel et al., EMBO J, 17, 1998: 4283-4290), and accordingly, serotonin and analogues thereof may be employed in the methods and compositions of the invention.

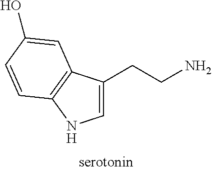

Examples of serotonin analogues are well known in the field and include, for example, 6-hydroxytetrahydro-beta-carboline and 6-hydroxy-3-aminotetrahydrocarbazole.

Analogues of the compounds suitable for use in the methods and compositions of the invention are also contemplated. The term “analogue” as used herein means a compound which comprises a chemically modified form of a specific compound or class thereof, and which maintains the pharmaceutical and/or pharmacological activities characteristic of said compound or class.

Other molecules that activate cAMP-induced phosphorylation of TREK-1 channels are thus also applicable for the therapeutic methods and compositions of the present invention. These include, but are not limited to, caffeine and theophylline, which exhibit IC₅₀ values of 377+/−54 mμM and 486+/−76 m μM, respectively, in TREK-1 channels expressed in Chinese hamster ovary cells (Harinath & Sikdar, Epilepsy Res, 2005, 64(3):127-35).

Inhibitors of the enzyme phosphodiesterase-IV (PDE-IV) also promote the synthesis of cAMP, and are thus be applicable for use in the methods and compositions of the present invention. PDE-IV inhibitors include, but are not limited to, imidazol-2-one and 2-cyanoiminoimidazole derivatives (Andres et al., Bioorg Med Chem Lett. 2002 Feb 25;12(4):653-8), 3-(3-cyclopentyloxy-4-methoxybenzyl)-6-ethylamino-8-isopropyl-3H purine hydrochloride (Gale et al., Br J Clin Pharmacol. 2002 November;54(5):478-84), denbufylline, nitraquazone, 9,10-Dimethoxy-2-mesitylimino-3-methyl-2,3,6,7-tetrahydro-4H-pyrimido-(6,1-a)-isoquinolin-4-one, rolipram and tibenelast (Spina et al., Life Sci. 1998;62(11):953-65), 3,5-Dimethyl-1-(3-nitrophenyl)-1H-pyrazole-4-carboxylic acid ethyl ester (Card, G. L., et al. 2005. Nat. Biotech. 23, 201).

Further compounds that act as inhibitors of TREK-1 include cationic ampathic molecules, a term which is used herein to described compounds that intercalate their hydrophobic ends primarily into the nonpolar interior of the lipid portion of the neuronal cell membrane bilayer, while their polar or ionic ends are exposed at the membrane-water interface (Patel et al., EMBO J, 1998, 17, 4283-4290). Such compounds include, but are not limited to, chlopromazine, above, as well as tetracaine:

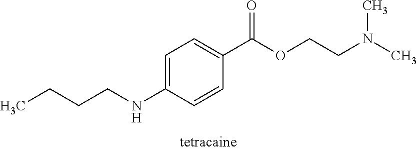

A further compound that acts as an inhibitor of stretch-activated TREK and TRAAK ion channels is GsTMx-4, a 35-mer peptide isolated from the venom of the spider Grammostola spatulata, which is also suitable for use in the methods and compositions of the invention (Suchyna et al., J Gen Physiol, 2000, 115:583-598). The equilibrium dissociation constant for GsTMx-4 is approximately 630 nM.

Compositions according to the invention may be formulated with standard buffers, excipients, carriers, diluents and the like. Examples of carriers include: water, physiologically saline, isotonic solutions containing dextrose, glycerol or other agents conferring isotonicity, lower alcohols, vegetable oils, polyethylene glycol, glycerol triacetate and other fatty acid glycerides. Examples of other carriers which may be used include cream forming agents, gel forming agents, and the like, compounding and tabletting agents. Excipients include buffers, stabilisers, emulsion forming agents, colouring compounds, salts, amino acids, antibiotics and other anti-bacterial compounds chelating agents and the like. More than one excipient and carrier may be used.

The amount or dosage of compounds used to protect neural tissue from pressure induced apoptotic cell death will depend upon various factors including the neural tissue to be treated, such as that in the eye, in the brain, or in peripheral tissue such as in the hand, leg, foot, fingers, oral cavity, nose or ear, the manner of delivery, the severity of the condition being treated, and the judgement of the prescribing physician. By way of example, compounds of the invention may be delivered as a solution for installation, such as an eye drop, ear drop, nose drop; an injectible sterile subcutaneous or intravenous solution; in the form of a tablet, capsule, suppository, dragee; or in the form of a transdermal composition; all of which are well known in the pharmaceutical field and described for example in Remington's Pharmaceutical Sciences Mack Publishing Company, Philadelphia. Generally, the concentration of active agents, which may be regarded as therapeutically effective, will be in the order of 0.001 M to 500 mM, such as from 0.1 M to 100 M, 50 M to 100 M, 100 M to 500 M, 500 M to 1 mM, or 1 mM to 500 mM.

Calculation of an appropriate dose of the compounds for use in the methods and compositions of the invention is a well-known art of pharmacology. In vitro data obtained, for example, from patch clamping studies may be used to calculate in vitro IC₅₀ values. These values are than extrapolated to appropriate in vivo dosages for animal trials using computer software, where the behaviour (ie pharmacokinetics) of the compound in question is analysed (ie dose-response relationships, biodistribution, excretion kinetics etc).

In vitro, cell-line experiments (eg patch clamping) determine the relationship between dose and inhibition of the stretch-activated ion channel, that is, what dose results in what degree of inhibition of channel activity. For example, chlorpromazine and mibefradil have been found to inhibit basal and lysophosphatidic acid-induced potassium currents in TREK-1 channels in vitro in mouse neuronal cells, both at a concentration of 10 M (Chemin et al., J Biol Chem 2005, 280(6): 4415-4421). GsMTx-4 has been found to reduce stretch-activated whole-cell currents in hypotonically swollen astrocytes by around 40% at a concentration of 5 M (Suchyna et al., J Gen Physiol, 2000, 115: 583-98). Quinidine is a potent in vitro blocker of TREK-1 at a concentration of 1 mM (Patel et al., EMBO J, 1998, 17(15): 4283-90). Chlorpromazine and tetracaine were found to inhibit TREK-1 channel activity in vitro in transfected COS cells at concentrations of 10 M and 100 M, respectively (Patel et al., 1988, above).

Drugs that have favorable profiles are moved into animal models, where the tolerability of the doses is assessed. Also determined at this point is the drug pharmacokinetics, how it is processed (absorption, distribution, metabolism and elimination) in the body, which can determine how frequently it should be dosed.

Phase I studies are then done to establish the tolerable dose in humans. These studies begin by administering low doses (determined by extrapolation from animal studies), then gradually increasing the doses in subsequent subjects, carefully observing for side effects (and effect on tumor to a lesser extent). Dosing is almost always based on a given number of milligrams of drug for every square meter of body surface area (BSA). The BSA is used to standardize the dose of drug delivery in patients of different heights and weights. A variety of computer programs exist that assist in each step of the dose-calculation process. (eg GraphPad)

In relation to the treatment of glaucoma, compositions of the invention may be administered to the eye, such as by way of eye drop or intraocular injection or as a systemic medication or oral dosage form such as a tablet etc.

The present invention in one of its aspects represents a significant advance in relation to the treatment of glaucoma. Theories of glaucoma pathogenesis to date are controversial and unclear. Whilst elevated pressure in the eye is a characteristic of glaucoma, the ways in which retinal ganglion cell death is mediated, and may be prevented, are unknown. The inventor's work indicates that pressure alone may be the stimulus for apoptosis in neuronal cells, both in culture and in vivo. Blocking the apoptotic effect of pressure on neuronal cells, such as by inhibiting stretch-activated channels, provides therapeutic outcomes.

Compositions for the treatment of glaucoma may be administered to a subject one or more times per day, on or alternative days as a single administration or on a weekly basis. It is preferred that the compositions are administered to the eye for the treatment of glaucoma on a daily basis, generally from 1 to 3 times per day, such as at 5 to 8 hour intervals.

In the treatment of elevated of apoptotic damage to neuronal cells of the central nervous system resulting from elevated pressure in the CNS, such as that due to hydrocephalus, compounds of the invention may be formulated by conventional means known in the art so as to cross the blood-brain barrier. Such compositions may be administered parenterally or non-parenterally as described above, such as by way of oral administration in the form of a capsule, table or the like, rectal or vaginal administration, intravenous administration or intramuscular administration.

In the treatment of pressure induced apoptotic neuronal cell death in peripheral nerves, administration of the compounds of the invention will depend upon the site of the neurons/condition being treated. By way of example, increased neuronal cell pressure in the spine may be treated by way of transdermally active compositions, intramuscular injection, intralumbar injection, intravenous administration, oral administration, rectal administration or inhalation administration.

In order that the invention may be readily understood and put into practical effect, particular preferred embodiments will now be described by way of the following non-limiting examples.

### EXAMPLE 1

Pressure-induced Primary Retinal Ganglion Cell (RGC) apoptosis is believed by the applicant to be mediated by stretch activated channels. Recently a stretch activated receptor has been identified in animal and human RGCs and their amino acid sequence determined. TRAAK is a mechanogated K⁺ channel, opened by membrane stretch and activated by arachidonic acid. The present inventor has confirmed the presence of TRAAK in the RGC-5 line and shown arachidonic acid induction of apoptosis. A second channel of relevance is TREK-1.

The RGC-5 cell line is a vector transformed neuronal line derived from primary rat RGC cultures. Developed by Prof N. Agarwal at the University of North Texas, Fort Worth, it has been characterized by morphology, cell markers and PCR analysis.

The pressure chamber based in-vitro system used in this experiment is as previously described by Agar A, Yip S S, Hill M A, Coroneo M T. “Pressure related apoptosis in neuronal cell lines” J Neurosci Res. 2000;60:495-503. This system determines neuronal apoptosis in response to elevations in ambient hydrostatic pressure. Experiments to date have exposed RGC-5 neurones to pressure conditions analogous to normal intra-ocular pressure (15 mm Hg), chronic glaucoma (30 mm Hg) and acute glaucoma (100 mm Hg). Compared to non-pressurized controls, increased proportions of apoptotic RGC-5 neurones have been found at all these pressure levels. Further, this effect increases with increasing pressure.

These stretch activated channels are from of a family of two-pore domain K⁺ channels (K_{2P}) of which 8 have been cloned in rodents and humans. These include TREK-1 and TRAAK stretch-activated potassium channels.

TRAAK appears to be restricted to the central nervous system, spinal cord and retina. TREK-1 is ubiquitous with strong expression in the central nervous system. Both TREK-1 and TRAAK are outward rectifier K⁺ channels opened by membrane stretch, cell swelling, and/or shear stress (all pressure effects). At atmospheric pressure, basal activity is negligible and channels are opened by convex curvature of the plasma membrane. Mechano-gating does not require the integrity of the cytoskeleton and the activating force is apparently directly coming from the cell membrane bilayer. Cytoskeleton disruption potentiates the opening by membrane stretch, suggesting that these channels are tonically repressed by the cytoskeleton.

Immunolocalisation by specific antibodies has shown that these two channels have different subcellular locations—whereas TRAAK is mainly present in soma and to a lesser degree in axons and dendrites, TREK-1 is concentrated in dendrites. Thus both channels are relevant in relation to retinal ganglion cell pressure responses in glaucoma. The present inventor confirmed the presence of TRAAK in both human retina and retinal cell lines of RGC-5.

The pressure to half-maximum activation derived from the above system is 36 mm Hg for TREK-1 and 46 mm Hg for TRAAK. This is of special significance since intraocular pressure of 30 mm Hg is clinically recognized to be the pressure above which retinal ganglion cell damage is highly likely—treatment to lower pressure is usually commenced when eye pressure is at 30 mm Hg or higher. It has also recently been shown that patients with carpal tunnel syndrome had a mean carpal canal pressure of 32 mm Hg (normal 9.6 mm Hg) (see Szabo R, Chidgey L K. J Hand Surg (Am). 1989;14:624). Thus it appears that, human neural tissue is sensitive to pressures of approximately 30 mm Hg.

To investigate the potential for pharmacomodulation of these ion channels the present inventor conducted experiments testing a blocker of such channels in a bioassay. Sipatrigine was been used experimentally to block the adverse effects of pressure induced apoptosis in retinal ganglian cells, and neural cells.

To date the inventor has used sipatrigine (80 microM/L, n=8) to inhibit pressure induced apoptosis in our RGC-5 cells as well as MT2 a neural cell line.

The present inventor has also shown that arachidonic acid (1, 10, 50, 100 microM/L, n=4 at each concentration) induces apoptosis in RGC-5. and MT2 neural cell lines and other cell lines, and that this effect is also blocked by sipatrigine, at a dose of approximately 8 μM.

These data taken together indicate that the effector mechanism for retinal ganglion and neural cell death involves stretch activated channels, principally TRAAK and TREK-1 and that blocking these channels would inhibit cell death in clinical conditions such as glaucoma and pressure induced neural damage.

### EXAMPLE 2

### Glaucoma Model in the Rat

There are a number of experimental animal models for human glaucoma including a recently established rat model in which chronic ocular hypertension is induced (WoldeMussie E, Ruiz G, Wijono M, Wheeler L A. Neuroprotective effect of Brimonidine in chronic ocular hypertensive rats. IOVS 2000; 41:S830). In this model intraocular pressures are elevated by laser photocoagulation of episcleral and limbal vessels (retarding the egress of aqueous humour from the eye), the levels of pressure being up to 2 fold in 2 to 3 weeks. This elevated pressure results in retinal ganglion cell death as occurs in glaucoma and 33±2.9% of retinal ganglion cells are lost in this model.

This model is used in tests. In a groups of experimental animals intraocular pressure is elevated and these animals are treated with systemic amiloride (20 mg/kg IP), gentamicin (10 mg/kg IP) or gadolinium (70 mg/kg IP).

Reduction in pressure-induced retinal ganglion cell loss compared untreated controls is observed, supporting the therapeutic treatments of this invention.

### EXAMPLE 3

### Human Studies

In humans, acute glaucoma is a condition in which there is a sudden rise of eye pressure, usually brought about by closure of the drainage angle of the eye (iris blocks the angle). Damage to the retinal ganglion cells and iris precedes damage to most other tissues in the eye. Despite treatment to lower eye pressure, significant and often severe retinal ganglion cell damage occurs.

Controlled studies are carried out using blockers of stretch-activated channels to reduce the severity of retinal ganglion cell damage in patients with acute glaucoma. All patients who are subject to conventional treatment to lower intraocular pressure as soon as diagnosis is made. They are then randomized to control and experimental groups. The experimental group is treated with systemic sipatrigine (10-200 mg/kg), or local sipatrigine or local chlorpromazine (by eyedrop), both potent inhibitors of TREK-1 and TRAAK channels that have been previously been safely used in the treatment of stroke and psychosis, respectively (see Dawson D A, Wadsworth G, Palmer A M. Brain Res. 2001;892:344-50). Better outcomes in retinal ganglion cell survival (and therefore field of vision) in the systemic and local sipatrigine and chlorpromazine-treated groups are achieved, thus forming the basis of using this invention in other forms of glaucoma or neural damage induced by pressure.

1. A method for the treatment of a condition associated with pressure-induced apoptotic neuronal cell death which comprises administering to a subject in need of such treatment at least one compound which directly or indirectly inhibits activity of a stretch-activated ion channel on neuronal cells.

2. The method of claim 1, wherein the at least one compound which inhibits activity of a stretch-activated ion channel on neuronal cells is identified by patch clamping.

3-8. (canceled)

9. The method of claim 1, wherein the neuronal cells are part of the central nervous system.

10. The method of claim 1, wherein the condition is glaucoma and the neuronal cells are intraocular neuronal cells.

11. The method of claim 1, wherein the neuronal cells are part of the peripheral nervous system.

12-22. (canceled)


SEQUENCE LISTING (2 sequences)

SEQ ID NO: 1 - protein, 426 aa, Homo Sapien [sequence omitted]

SEQ ID NO: 2 - protein, 419 aa, Homo Sapien
MTTAPQEPPARPLQAGSGAGPAPGRAMRSTTLLALLALVLLYLVSGALVFRALEQPHEQQAQRELGEVREKFLRAHPCVSDQELGLLIKEVADALGGGADPETNSTSNSSHSAWDLGSAFFFSGTIITTIGYGNVALRTDAGRLFCIFYALVGIPLFGILLAGVGDRLGSSLRHGIGHIEAIFLKWHVPPELVRVLSAMLFLLIGCLLFVLTPTFVFCYMEDWSKLEAIYFVIVTLTTVGFGDYVAGADPRQDSPAYQPLVWFWILLGLAYFASVLTTIGNWLRVVSRRTRAEMGGLTAQAASWTGTVTARVTQRAGPAAPPPEKEQPLLPPPPCPAQPLGRPRSPSPPEKAQPPSPPTASALDYPSENLAFIDESSDTQSERGCPLPRAPRGRRRPNPPRKPVRPRGPGRPRDKGVPV